(19) United States
(12) Patent Application Publication
(10) Pub. No.: US 2024/0174583 A1
(43) Pub. Date: May 30, 2024

(54) PROCESSES FOR THE VAPOR PHASE HYDROGENATION OF ALDEHYDES

(71) Applicant: Dow Technology Investments LLC, Midland, MI (US)

(72) Inventors: Glenn A. Miller, South Charleston, WV (US); Jin Yang, Pearland, TX (US)

(21) Appl. No.: 18/550,297

(22) PCT Filed: Apr. 6, 2022
(86) PCT No.: PCT/US2022/023600

§ 371 Date: Sep. 13, 2023

Related U.S. Application Data
(60) Provisional application No. 63/192,901, filed on May 25, 2021.

(51) Int. Cl. C07C 29/141

(52) U.S. Cl. CPC C07C 29/141

(57) ABSTRACT

The present invention relates to processes for the vapor phase hydrogenation of aldehydes. In one embodiment, the process comprises (a) providing a liquid aldehyde stream to a vaporization system to generate a vaporous aldehyde stream in the presence of a weakly basic amine, wherein the weakly basic amine has a normal boiling point that is at least 50 C greater than the normal boiling point of the aldehyde, wherein the weakly basic amine reacts with acidic impurities in the liquid aldehyde stream to form ammonium salt adducts, and wherein the ammonium salt adducts and any excess weakly basic amine are removed as a heavies purge from the vaporization system; (b) combining the vaporous aldehyde stream with a hydrogen stream by either providing a hydrogen stream to the vaporization system, by adding a hydrogen stream to the vaporous aldehyde stream following step (a), or by a combination thereof; (c) providing the combined vaporous aldehyde and hydrogen stream to a vapor phase hydrogenation zone; and (d) hydrogenating the vaporous aldehyde in the vapor phase hydrogenation zone.

### FIELD

The present invention relates generally to processes for the vapor phase hydrogenation (VPH) of aldehydes.

### BACKGROUND

The use of heterogeneous (packed bed) hydrogenation catalysts for the reduction of aldehydes (and unsaturated aldehydes) to the corresponding alcohols is well known. In utilizing such hydrogenation catalysts, especially in vapor phase hydrogenation, a number of issues need to be considered including, for example, reactivity, selectivity (avoiding side-reactions), and pressure drop across the bed.

For example, considerable work has been done in regards to the shape and size of the hydrogenation catalyst particle to mitigate these issues. U.S. Pat. No. 4,673,664 discusses improvement of pressure in fixed bed reactors by using helical, lobed, or polylobed catalyst particles formed by extrusion that create additional void space due to their ornate structures. US Patent Publication No. 2017/0189875 discusses improvement of pressure drop in fixed bed reactors by using catalyst particles of various ornate shapes, and discusses the reactor design employing these particles.

U.S. Pat. No. 6,096,931 teaches adding low levels (1 to 50 ppm by nitrogen) of amines to the vapor phase entering the VPH catalyst zone to modify behavior of the VPH catalyst itself, presumably by modifying sites on the catalyst itself. In order to achieve the required levels of amine in the vapor phase, the amines must be volatile or the vaporization and VPH temperatures must be high enough to vaporize the amine and avoid condensation on the VPH catalyst. This process then requires separating the amine after the VPH reaction zone. Having the amine present in the VPH zone and the downstream refining processes is likely to generate heavies by the amine-catalyzed aldol condensation. This reference is silent on the impact of acidic species coming in with the aldehyde feed and offers no remedy for incoming acidity.

Another issue that needs to be considered is degradation of solid hydrogenation catalysts used in vapor phase hydrogenation. As the catalyst is used, it has been found that the solid catalyst degrades to generate “fines” or “catalyst dust.” The exact nature of these “fines” is undefined and may vary depending on the nature of the catalyst and the support. However, the ability of the catalyst to tolerate the generation of these fines has not been adequately addressed in the past. The generation of fines from the hydrogenation catalyst can lead to undesirable increases in pressure drop across the catalyst bed. A catalyst bed that starts off with excellent performance but degrades rapidly (i.e., exhibits rapid pressure drop increase with time) will need to be replaced frequently which may require a plant shutdown, expensive catalyst recovery, or catalyst disposal.

It would be desirable to have processes for the vapor phase hydrogenation of aldehydes that minimize catalyst degradation and thus improves catalyst.

### SUMMARY

In a typical vapor phase hydrogenation (VPH) process, prior to entering the hydrogenation reactor, an aldehyde stream passes through a vaporizer. It has been found that having a mild base present during the vaporization process will mitigate at least one cause of catalyst degradation with some catalyst supports. It is believed that the mild bases neutralize acidic impurities that may be present in the aldehyde stream (e.g., carboxylic acids presumably derived from olefin carbonylation, oxidation of the aldehyde, or heavies ester hydrolysis). In addition, the presence of acidic impurities may cause side reactions during the vaporization and hydrogenation processes. For example, in many cases, the amount of aciditc impurities from an upstream hydroformylation process (including any interim storage) may be sufficiently high to cause degradation of the VPH catalyst as well as cause side reactions resulting in a reduced yield.

In one embodiment, a process for the vapor phase hydrogenation of aldehydes comprises:
(a) providing a liquid aldehyde stream to a vaporization system to generate a vaporous aldehyde stream in the presence of a weakly basic amine, wherein the weakly basic amine has a normal boiling point that is at least 50° C. greater than the normal boiling point of the aldehyde, wherein the weakly basic amine reacts with acidic impurities in the liquid aldehyde stream to form ammonium salt adducts, and wherein the ammonium salt adducts and any excess weakly basic amine are removed as a heavies purge from the vaporization system;
(b) combining the vaporous aldehyde stream with a hydrogen stream by either providing a hydrogen stream to the vaporization system, by adding a hydrogen stream to the vaporous aldehyde stream following step (a), or by a combination thereof;
(c) providing the combined vaporous aldehyde and hydrogen stream to a vapor phase hydrogenation zone; and
(d) hydrogenating the vaporous aldehyde in the vapor phase hydrogenation zone.

These and other embodiments are described in more detail in the Detailed Description.

### BRIEF DESCRIPTION OF THE FIGURE

FIG. 1 is a system diagram illustrating process streams and equipment used according to one embodiment of the present invention.

### DETAILED DESCRIPTION

This disclosure relates generally to processes for the vapor phase hydrogenation of aldehydes. VPH typically involves contacting at least one aldehyde with hydrogen under heterogeneous VPH conditions sufficient to form at least one alcohol product in the presence of a fixed bed catalyst comprising, as components, a transition metal and at least one support. Prior to hydrogenation, a liquid aldehyde stream is typically converted to a vaporous aldehyde stream in a vaporization system. In one aspect, the present invention comprises providing a liquid aldehyde stream to a vaporization system to generate a vaporous aldehyde stream in the presence of a weakly basic amine, wherein the weakly basic amine has a normal boiling point that is at least 50° C. greater than the normal boiling point of the aldehyde, wherein the weakly basic amine reacts with acidic impurities in the liquid aldehyde stream to form ammonium salt adducts, and wherein the ammonium salt adducts and any excess weakly basic amine are removed as a heavies purge from the vaporization system. The vaporous aldehyde stream is combined with a hydrogen stream by either providing a hydrogen stream to the vaporization system, by adding a hydrogen stream to the vaporous aldehyde stream following vaporization, or by a combination thereof. The combined vaporous aldehyde and hydrogen stream is provided to a vapor phase hydrogenation zone, and the vaporous aldehyde is hydrogenated in the vapor phase hydrogenation zone.

All references to the Periodic Table of the Elements and the various groups therein are to the version published in the CRC Handbook of Chemistry and Physics, 72nd Ed. (1991-1992) CRC Press, at page I-11.

Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims. Thus, for example, an aqueous composition that includes particles of “a” hydrophobic polymer can be interpreted to mean that the composition includes particles of “one or more” hydrophobic polymers.

Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.01 to 100, from 1 to 99.99, from 1.01 to 99.99, from 40 to 60, from 1 to 55, etc. Also herein, the recitations of numerical ranges and/or numerical values, including such recitations in the claims, can be read to include the term “about.” In such instances the term “about” refers to numerical ranges and/or numerical values that are substantially the same as those recited herein.

As used herein, the term “ppmw” means parts per million by weight. When used to assess concentration of weakly basic amine, the phrase “ppmw (by nitrogen)” is based on the weight of the amine nitrogen divided by the total weight of the mixture. This makes the analysis independent of the molecular weight of the amine and focuses on the active group on the weakly basic amine. The amine nitrogen does not include nitrogen moieties not capable of reacting with acids such as quaternary amines.

As used herein, the term “substituted” is contemplated to include all permissible substituents of organic compounds unless otherwise indicated. In a broad aspect, the permissible substituents include acyclic and cyclic, branched and unbranched, carbocyclic and heterocyclic, aromatic and nonaromatic substituents of organic compounds. Illustrative substituents include, for example, alkyl, alkyloxy, aryl, aryloxy, hydroxyalkyl, aminoalkyl, in which the number of carbons can range from 1 to 20 or more, preferably from 1 to 12, as well as hydroxy, halo, and amino. The permissible substituents can be one or more and the same or different for appropriate organic compounds. This invention is not intended to be limited in any manner by the permissible substituents of organic compounds.

As used herein, the term “vapor phase hydrogenation” is contemplated to include, but is not limited to, all vapor phase hydrogenation processes that involve converting one or more substituted or unsubstituted aldehyde compounds or a reaction mixture comprising one or more substituted or unsubstituted aldehyde compounds to one or more substituted or unsubstituted alcohols or a reaction mixture comprising one or more substituted or unsubstituted alcohols using a heterogeneous (solid) catalyst. The alcohols may be asymmetric or non-asymmetric. The starting aldehyde may be unsaturated (conjugated or not conjugated with the aldehyde moiety), and the resulting product may be the corresponding saturated or unsaturated alcohol.

In one aspect, a process for the vapor phase hydrogenation of aldehydes comprises: (a) providing a liquid aldehyde stream to a vaporization system to generate a vaporous aldehyde stream in the presence of a weakly basic amine, wherein the weakly basic amine has a normal boiling point that is at least 50° C. greater than the normal boiling point of the aldehyde, wherein the weakly basic amine reacts with acidic impurities in the liquid aldehyde stream to form ammounium salt adducts, and wherein the ammonium salt adducts and any excess weakly basic amine are removed as a heavies purge from the vaporization system; (b) combining the vaporous aldehyde stream with a hydrogen stream by either providing a hydrogen stream to the vaporization system, by adding a hydrogen stream to the vaporous aldehyde stream following step (a), or by a combination thereof; (c) providing the combined vaporous aldehyde and hydrogen stream to a vapor phase hydrogenation zone; and (d) hydrogenating the vaporous aldehyde in the vapor phase hydrogenation zone.

Simple amines such as trialkylamines are too basic but simple alkanolamines and and heterocyclic nitrogen compounds such as imidazoles are sufficiently low in basicitiy to effectively neutralize the acidic impurities without generating aldol condensation heavies. This translates to a longer catalyst life. In addition, the removal of the feed acids will contribute to the reduction of side reactions in the VPH process itself. Because the alkanolamines and imidizoles exhibit very low volatility, they do not vaporize into the VPH system and are removed (with any salts) in the vaporizer heavies stream and thus there is no impact on downstream refining. The alkalinity or basicity of the weakly basic amine is generally reported as the pKa of the conjugate acid, which advantageously is from 5 to 11 at 25° C. The pKa, in some embodiments, is preferably from 5.0 to 9.5 at 25° C. and most preferably is from 6.0 to 9.0 at 25° C. In some embodiments, the weakly basic amine has a normal boiling point that is at least 100° C. greater than the normal boiling point of the aldehyde. In some embodiments, the weakly basic amine used in process of the present invention comprises a trialkanolamine or an imidazole. In some embodiments, the weakly basic amine used in processes of the present invention comprises triethanolamine or benzimidazole.

In some embodiments, the concentration of the weakly basic amine in the combined vaporous aldehyde and hydrogen stream in step (c) (the combined stream provided to the vapor phase hydrogenation zone) is less than 1 ppmw (by nitrogen).

In some embodiments, processes of the present invention further comprise measuring the acid content of the liquid aldehyde stream, and the amount of weakly basic amine added to the vaporization system is between 0.1 and 5 equivalents of weakly basic amine to equivalents of acid. In some embodiments, the amount of weakly basic amine added to the vaporization system is between 0.1 and 1.5 equivalents of weakly basic amine to equivalents of acid.

In some embodiments, the liquid aldehyde stream that is vaporized and hydrogenated is provided from a hydroformylation reaction and a product-catalyst separation step, wherein hydroformylation catalyst is separated from a hydroformylation product stream in the product-catalyst separation step to provide the liquid aldehyde stream.

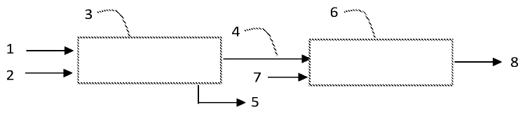

FIG. 1 is a system diagram illustrating process streams and equipment used according to one embodiment of the present invention. As shown in FIG. 1, a liquid aldehyde stream 1 is fed to a vaporization system 3, optionally with a hydrogen gas stream 2. The volatilized aldehyde (with optional H₂) leaves the vaporization device 3 via stream and any non-volatilized material leaves via stream 5. If hydrogen gas is not supplied to the vaporization system, in some embodiments, hydrogen gas can be added to the vaporous aldehyde stream 4 leaving the vaporization device 3 or to the VPH unit 6 (discussed below). Stream 4 with vaporous aldehyde and any hydrogen added to the vaporization system 3 is then subjected to vapor phase hydrogenation in a vapor phase hydrogenation zone in a VPH unit 6. In some embodiments, hydrogen may also be provided to the VPH unit 6 via stream 7 (or combined with stream 4 before entering the VPH unit). To be clear hydrogen may be provided to the VPH unit 6 in a number of ways: (a) hydrogen may be added to the vaporization system 3 via stream 2 and then exit with the vaporous aldehyde in stream 4; (b) hydrogen may be added to the VPH unit 6 as a separate stream 7; (c) hydrogen may be added to stream 4 prior to entering the VPH unit 6; or any combination of (a), (b), and (c). The crude alcohol product and excess H₂, unconverted aldehyde, and gaseous inerts leave via stream 8 for further processing including separating unreacted H₂ and/or aldehyde and recirculating one or both of them back to a previous unit. For example, unreacted hydrogen can be separated and recycled back to the process as part of stream 2, stream 7, or another stream. Recycle streams either to the vaporization system 3 or the VPH unit 6 are not shown for clarity. The weakly basic amine that is used in processes of the present invention would typically be added to stream 1 before the vaporization system 3 to allow good mixing, but could also be added to the vaporization system 3 directly.

The liquid aldehyde stream 1 can be directly from a hydroformylation unit or may comprise recycle streams or aldehydes from other unit operations (e.g., recycled from aldol condensation or from refining units). Any of these processes may generate acidic species that need to be removed prior to the aldehyde stream entering the vapor phase hydrogenation zone (e.g., contacting the VPH catalyst).

The vaporization system 3 can be a simple distillation tower, a spray vaporizer, a thin-film vaporizer, a hydrogen stripping system, or a combination of these. If the weakly basic amine is not added to the liquid aldehyde stream, the weakly basic amine can be added to a different tray in a distillation tower (in those embodiments where the vaporization system is a distillation tower) from the liquid aldehyde feed, typically a few trays below the top tray, or as part of the reflux flow, or any combination of these.

The vaporization system should have a heavies removal stream (e.g., stream 5 in FIG. 1) to remove heavies formed during the hydroformylation process and during storage prior to being fed to the vaporizer system. This process also removes hydroformylation catalyst residues (either entrained or sublimed) and protects the VPH catalyst from condensation by heavy organics. The weakly basic amines and any salts they form with incoming acidic species will be removed with the heavies in this purge. The resulting purge can be processed further to recover any valuable aldehyde to be recycled and possibly to recover weakly basic amines for reuse.

Hydrogen is required for the vapor phase hydrogenation of aldehyde and may be provided to a VPH unit as described. Hydrogen may be obtained from any suitable source, including petroleum cracking and refinery operations.

The nature and composition of aldehyde vapor phase hydrogenation catalysts are well known. The catalysts useful in a vapor phase hydrogenation process comprise a catalytic metal on a support. The catalytic metal can include Group 8, 9 and 10 metals selected from rhodium (Rh), cobalt (Co), iridium (Ir), ruthenium (Ru), iron (Fe), nickel (Ni), palladium (Pd), platinum (Pt), chromium (Cr), osmium (Os), Copper (Cu), and mixtures thereof, with preferred metals being palladium, platinum, copper, and nickel.

Catalyst supports for vapor phase hydrogenation catalysts are generally inert, solid materials designed to hold the active catalyst metal. Examples include graphite, activated carbon, silica, alumina, and metal oxides such as molybdenum oxide, chromium oxide, zinc oxide, titania, and the like. Supports may be composed of a combination of different materials and other additives which offer different properties such as improved crush strength, reduced metal leaching, reduced side products, and ease of extrusion among others.

As an illustration, the catalytic metel may be impregnated onto any solid support, such as inorganic oxides, (i.e., alumina, silica, titania, or zirconia) carbon, or ion exchange resins. The catalyst may be supported on, or intercalated inside the pores of, a zeolite, glass or clay; the catalyst may also be dissolved in a liquid film coating the pores of said zeolite or glass. Such zeolite-supported catalysts are particularly advantageous for producing one or more regioisomeric alcohols in high selectivity, as determined by the pore size of the zeolite. The techniques for supporting catalysts on solids, such as incipient wetness, which will be known to those skilled in the art. The solid catalyst thus formed may still be complexed with one or more of the ligands defined above. Descriptions of such solid catalysts may be found in for example: J. Mol. Cat., 1991, 70, 363-368; Catal. Lett., 1991, 8, 209-214; J. Organomet. Chem., 1991, 403, 221-227; Nature, 1989, 339, 454-455; J. Catal., 1985, 96, 563-573; J. Mol. Cat., 1987, 39, 243-259.

The nature of the support is not narrowly critical for the present invention but it has been observed that some supports are more vulnerable than others to acid degradation. Zinc oxide in particular appears to be vulnerable whereas chromium oxide is less so. Acidic impurities may degrade the support, facilitate metal leaching, or change the nature of the catalyst surface or pore structure.

It should be noted that the exact composition and microscopic (pore) structure of the heterogeneous vapor phase hydrogenation catalyst is not narrowly critical to the present invention which is primarily dealing with changes in the catalyst nature. The most obvious observed issues are a change in catalyst performance (e.g., hydrogenation rate, selectivity, pressure drop, and hot-spot location within the bed). The increase in pressure drop is often caused by the generation of fines. The nature of the fines, how they are generated, and how they migrate in the bed are also not narrowly critical other than it is observed and changes in pressure drop are observed to the degree that catalyst performance and/or catalyst bed performance is negatively impacted.

To determine whether metal leaching or the generation of fines has occurred during vapor phase hydrogenation, the catalytic metal concentration in the catalyst and/or in the condensed liquid product can be measured using analytical techniques well known to those having ordinary skill in the art, such as atomic absorption (AA), inductively coupled plasma (ICP), X-ray diffraction (XRD), and X-ray fluorescence (XRF) are typically preferred.

One indicator of whether there may be issues with the catalyst (e.g., generation of fines) is the pressure drop across the vapor phase hydrogenation reactor, such that, in some embodiments, the pressure drop across the hydrogenation reaction can be monitored. Pressure drop as used herein refers to the difference in pressure between the feed point of the hydrogenation reactor (typically measured at or near the aldehyde feed point) and the exit point of the reactor. As the reaction materials pass through the heterogeneous catalyst, the flow encounters resistance due to the catalyst which results in a drop in pressure as the materials flow through the bed. Excessive pressure drop can result in further catalyst bed degradation (e.g., crushing or abrasion) and in the case of vapor phase hydrogenation, possible condensation, channeling, and heat transfer issues. Particle fines tend to increase flow resistance thus are a major contributor to pressure drop increases with time and can lead to the need to change out the catalyst. The exact nature of the fines and how they are generated is often not known but are usually attributed to catalyst fracturing, abrasion, chemical/physical erosion (leaching) and the like.

It is known that fines can be generated at the start of the catalyst life such as during the initial catalyst loading. For the purposes of this invention, the increase in pressure drop can be monitored after the initial “break in” period. After the initial “break-in” period, the pressure drop remains steady for a period of time and then begins to increase with time often in an exponential manner.

The critical pressure drop value will differ depending on the catalyst system and equipment of course but when the catalyst reactor efficiency and operation is impacted by the pressure drop, it becomes an economic decision as to whether to continue at sub-optimal performance (e.g., lower rates, lower conversion, higher side products, duplicate/swinging reactor) or to stop operation and change out the catalyst. Embodiments of the present invention can advantageously extend the life of the catalyst bed and thus postpone and/or reduce the costs of catalyst purchase, a plant shutdown, and associated catalyst precious metal recovery or disposal.

For the purposes of this invention, the term “weakly basic amine” encompasses relatively non-volatile substutituted amines and heterocyclic nitrogen compounds as described below. The weakly basic amines function as acid scavengers or acidity mitigation agents to remove the acidic components from the aldehyde feed stream in the vaporization system as an adduct (typically a salt) in the vaporizer bottom purge. While it may be preferred to employ only one weakly basic amine species at a time in any given VPH process, if desired, mixtures of two or more different weakly basic amine species may also be employed in any given process. By adding weakly basic amine to the vaporization system, acidic components are advantageously removed prior to the hydrogenation zone where such components can cause degradation of the hydrogenation catalyst as discussed herein.

The weakly basic amine useful in embodiments of the present invention advantageously has the following two properties: (1) it is weakly basic in order to avoid heavies formation in the vaporization system; and 2) it is non-volatile in order to avoid contacting and collecting (condensation) on the VPH catalyst under hydrogenation conditions. The alkalinity or basicity of the weakly basic amine is generally reported as the pKa of the conjugate acid, which advantageously is from 5 to 11 at 25° C. The pKa is preferably from 5.0 to 9.5 at 25° C. in some preferred embodiments and from 6.0 to 9.0 at 25° C. in other preferred embodiments. In some embodiments, the weakly basic amines are not strong facilitators of the formation of heavies. In some embodiments, weakly basic amines can be tested for heavies formation by heating the product aldehyde with the weakly basic amine at elevated temperature such as at or near the vaporization temperature of the aldehyde. In some embodiments, the weakly basic amines will exhibit less than 1 gram of heavies formation per liter of test solution (product aldehyde+weakly basic amine in solution with the weakly basic amine typically added at a concentration of 1000 ppmw) per day at the vaporization temperature of the aldehyde. The amount of heavies formation can be readily determined by gas or liquid chromatography, as is known to those skilled in the art.

Regarding volatility, the volatility of the weakly basic amine should be such that under vaporization conditions for the vapor phase aldehyde, less than 1% of the weakly basic amine added is volatilized, preferably less than 0.1%, and most preferably less than 0.01%. This can be controlled by selecting a weakly basic amine with a normal boiling point at least 50° C. higher than the aldehyde to be hydrogenated in some embodiments, and with a normal boiling point at least 100° C. higher than the aldehyde in other embodiments.

The amount of weakly basic amine that is volatilized under vaporization conditions can also be managed by controlling the concentration of the weakly basic amine such that the amount of excess weakly basic amine (relative to the acidic impurities) reduces the “free” amine to low levels. If there is little free amine present, this will reduce the partial pressure of the amine and lower losses of the amine via the vaporized stream. The term “free amine” refers to amine that is not neutralized or reacted with the acidic impurities. For example, at an equimolar ratio of acid and amine, very little free amine is present in solution thus the amine partial pressure will be extremely low. This also means that the amount of free acid is also extremely low.

The amount of weakly basic amine employable in any given process of the present invention need only be that minimum amount necessary to furnish the basis for at least some minimization of catalyst decomposition as might be found to occur as a result of carrying out an identical metal catalyzed hydrogenation process under essentially the same conditions, but in the absence of any of the weakly basic amine during harsh conditions, such as vaporization separation of the aldehyde product. Thus, in some embodiments, the amount of weakly basic amine added to the vaporization system is between 0.1 and 5 equivalents of weakly basic amine to equivalents of acid in the liquid aldehyde stream provided to the vaporization system. In some embodiments, the amount of weakly basic amine added to the vaporization system is between 0.5 and 1.5 equivalents of weakly basic amine to equivalents of acid in the liquid aldehyde stream provided to the vaporization system. The acid content of the liquid aldehyde stream provided to the vaporization system is measured by titration.

The weakly basic amines that can be used in various embodiments of the present invention advantageously are selected from one or more of the following classes.

One class of weakly basic amine has the structure:

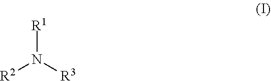

wherein R¹, R², and R³ each independently represent alkyl or aryl substituents such that none of R¹, R², and R³ is hydrogen, and wherein at least one has is an electron withdrawing substituent (either alpha or beta to the nitrogen moiety) and preferably at least 2 are electron withdrawing substituents. The electron withdrawing alkyl or aryl substituents include alkyl-substituted or unsubstituted aryl, alkoxylated, alkylalkoxylated, or carboxylated aryl groups, beta-alkoxy or beta-alkoxyalkyls (such as beta-hydroxyethyl, beta-hydroxy-alpha-methylethyl, beta-hydroxy-beta-methylethyl and ethoxylated and/or propoxylated adducts thereof). Examples of preferred amines of this class include triethanolamine, methyldiethanolamine, ethyldiethanolamine, dimethylethanolamine and tri(2-hydroxypropyl)amine and ethoxylates thereof. The preferred amines are trialkanolamines such as triethanolamine and tri(2-hydroxypropyl)amine.

A second class of weakly basic includes heterocyclic nitrogen compounds such as described in PCT Publication No. WO2019/083700. Such heterocyclic nitrogen compounds are well known, as are methods for their preparation. In many instances, such heterocyclic nitrogen compounds are readily available commercially. Suitable substituted and unsubstituted heterocyclic nitrogen compounds include those permissible substituted and unsubstituted heterocyclic nitrogen compounds described in Kirk-Othmer, “Encyclopedia of Chemical Technology,” Fourth Edition, 1996, the pertinent portions of which are incorporated herein by reference.

Illustrative heterocyclic nitrogen compounds that can be used as weakly basic amines in some embodiments of the present invention include the following diazoles:
(a) imidazoles represented by the formula:

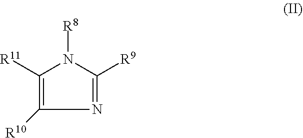

(b) pyrazoles represented by the formula:

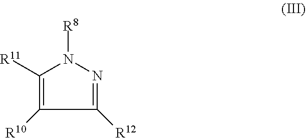

and (c) indazoles represented by the formula:

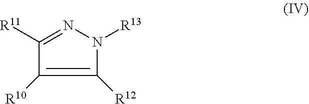

wherein in Formulas (II), (III) and (IV) above, R⁸, R⁹, R¹⁰, R¹¹, R¹² and R¹³ are identical or different and each represents a hydrogen atom or a monovalent substituent, with the proviso that, in one embodiment of the invention, R⁸ and R⁹ should not both be monovalent hydrocarbon radicals at the same time. The adjacent substituents R⁸ and R¹¹, or R⁸ and R⁹, or R¹⁰ and R¹¹, or R¹⁰ and R¹², or R¹² and R¹³ may optionally be taken together to form a substituted or unsubstituted divalent radical which together with the two atoms of the formula to which said adjacent substituents are bonded form a cyclic ring.

The monovalent R⁸ to R¹³ substituents in Formulas (II), (III) and (IV) can be any substituent that does not unduly adversely affect the purpose and process of the invention. Examples of such monovalent substituents include hydroxy, cyano, nitro, trifluoromethyl and substituted or unsubstituted radicals containing from 1 to 30 carbon atoms selected from the group consisting, acyl, acyloxy carbonyloxy, oxycarbonyl, silyl, alkoxy, aryloxy, cycloalkoxy, alkyl, aryl, alkaryl, aralkyl, and alicyclic radicals.

More specifically illustrative monovalent substituents containing from 1 to 30 carbon atoms include e.g., primary, secondary and tertiary alkyl radicals such as methyl, ethyl, n-propyl, isopropyl, butyl, sec-butyl, t-butyl, neo-pentyl, n-hexyl, arnyl, sec-amyl, t-amyl, iso-octyl, decyl, octadecyl, and the like; aryl radicals such as phenyl, naphthyl and the like; aralkyl radicals such as benzyl, phenylethyl, triphenylmethyl, and the like; alkaryl radicals such as tolyl, xylyl, and the like; alicyclic radicals such as cyclopentyl, cyclohexyl, 1-methylcyclohexyl, cyclooctyl, cyclohexylethyl, and the like; alkoxy radicals such as methoxy, ethoxy, propoxy, t-butoxy-OCH₂CH₂OCH₃, —O(CH₂CH₂)₂CH₃, —O(CH₂CH₂)₃OCH₃, and the like; aryloxy radicals such as phenoxy and the like; as well as silyl radicals such as —Si(CH₃)₃, —Si(OCH₃)₃, —Si(C₃H₇)₃, and the like; acyl radicals such as —C(O)CH₃, —C(O)C₂H₅, —C(O)C₆H₅, and the like; carbonyloxy radicals such as —C(O)OCH₃ and the like; oxycarbonyl radicals such as —O(CO)C₆H₅, and the like.

If desired, such monovalent substituents may in turn be substituted with any substituent which does not unduly adversely affect the purpose and process of this invention such as, for example, those hydrocarbon and non-hydrocarbon substituents outlined herein for R⁸, R⁹, R¹⁰, R¹¹, R¹² and R¹³. Formulas (II) through (IV) are also intended to encompass compounds having two or more such diazole formulas, e.g., wherein two diazole formulas are directly bonded together as a result of any one of the R⁸ to R¹³ substituents optionally representing a direct bond or as a result of any one of the R⁸ to R¹³ substituents being optionally substituted with a second diazole formula.

Moreover, said adjacent substituents, R⁸ and R¹¹, or R⁸ and R⁹, or R¹⁰ and R¹¹, or R¹⁰ and R¹², or R¹² and R¹³, may be taken together to form a substituted or unsubstituted divalent bridging group having from 3 to 5, preferably 4, carbon atoms, which along with the two atoms shown in the formula to which they are bonded, form a 5 to 7 membered cyclic ring. Such divalent bridging groups preferably consist of only carbon atoms, but may contain from 1 to 2 nitrogen atoms in addition to said carbon atoms. Examples of substituents that may be on the substituted divalent bridging groups are the same hydrocarbon and non-hydrocarbon substituents as those defined herein for R⁸, R⁹, R¹⁰, R¹¹, R¹² and R¹³. Preferred diazoles are the imidazoles of Formula (II) above, especially benzimidazoles.

Illustrative heterocyclic nitrogen compounds that can be used as weakly basic amines in some embodiments of the present invention also include triazole compounds such as the following:
(a) 1,2,3-triazoles represented by the formula:

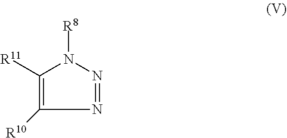

(b) 1,2,4-triazoles represented by the formula:

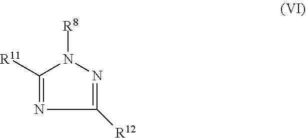

(c) 2,1,3-triazoles represented by the formula:

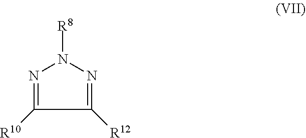

and (d) 4,1,2-triazoles represented by the formula:

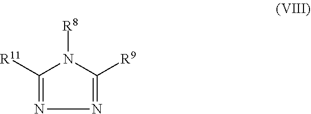

wherein in Formulas (V), (VI), (VII), and (VIII) above, R⁸, R⁹, R¹⁰, R¹¹ and R¹² are identical or different and each represents a hydrogen atom or a monovalent substituent, and adjacent substituents R⁸ and R⁹, or R⁸ and R¹¹, or R¹⁰ and R¹¹, or R¹⁰, and R¹², may optionally be taken together to form a substituted or unsubstituted divalent radical which together with the two atoms of the formula to which said adjacent substituents are bonded form a cyclic ring. More specifically said monovalent substituents of R⁸, R⁹, R¹⁰, R¹¹ and R¹² and the adjacent substituents R⁸ and R⁹, R⁸ and R¹¹, R¹⁰ and R¹¹, or R¹⁰ and R¹², in Formulas (V) to (VIII) above, may be the same as the monovalent substituents and divalent radicals defined for Formulas (II) to (IV) above. It is to be further understood that Formulas (V) through (VIII) are also intended to encompass compounds having two or more such triazole formulas, e.g., wherein two triazole formulas are directly bonded together as a result of any one of the R⁸, R⁹, R¹⁰, R¹¹ and R¹² substituents optionally representing a direct bond or as a result of any one of the R⁸, R⁹, R¹⁰, R¹¹ and R¹² substituents being optionally substituted with a second triazole formula. Preferred triazoles are the 1,2,3-triazoles of Formula (VIII) above, especially benzotriazole. Other illustrative triazoles include 5-methyl-1H-benzotriazole, 5,6-dimethyl-1-H-benzotriazole, 1-hydroxybenzotriazole, 2-(2H-benzotriazole-2-yl)-4-(1,1,3,3-tetramethylbutyl)-phenol, 5-nitrobenzotriazole, bis(1-benzotriazolyl) oxalate, 1-benzotriazolyl 9-fluorenylmethyl carbonate, 1-cyanobenzotriazole, 2-(2H-benzotriazol-2-yl)-hydroquinone, 2-(2-hydroxy-5-methylphenyl)-benzotriazole, 5-hexylbenzotriazole, 5-decylbenzotriazole, 1-ethylbenzotriazole, 1-pentylbenzotriazole, 1-benzylbenzotriazole, 1-dodecylbenzotriazole, and the like.

Illustrative heterocyclic nitrogen compounds that can be used as weakly basic amines in some embodiments of the present invention also include diazine compounds such as the following:
(a) 1,2-diazines represented by the formula:

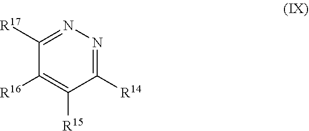

(b) 1,3-diazines represented by the formula:

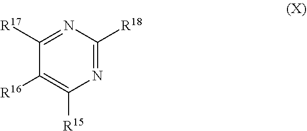

and (c) 1,4-diazines represented by the formula:

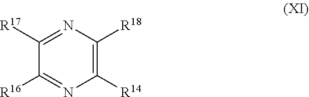

wherein in Formulas (IX), (X) and (XI) above, R¹⁴, R¹⁵, R¹⁶, R¹⁷ and R¹⁸ are identical or different and each represents a hydrogen atom or a monovalent substituent, and adjacent substituents R¹⁴ and R¹⁵, or R¹⁵ and R¹⁶, or R¹⁶ and R¹⁷, or R¹⁴ and R¹⁸ may optionally be taken together to form a substituted or unsubstituted divalent radical which together with the two atoms of the formula to which said adjacent substituents are bonded form a cyclic ring. More specifically, said monovalent substituents R¹⁴, R¹⁵, R¹⁶, R¹⁷ and R¹⁸, and the adjacent substituents R¹⁴ and R¹⁵, or R¹⁵ and R¹⁶, or R¹⁶ and R¹⁷, or R¹⁴ and R¹⁸, in Formulas (IX) to (XI) above, may be the same as the monovalent substituents and divalent radicals defined for Formulas (II) to (IV) above. It is to be further understood that Formulas (IX) through (XI) are also intended to encompass compounds having two or more such diazine formulas, e.g., wherein two diazine formulas are directly bonded together as a result of any one of the R¹⁴ to R¹⁸ substituents optionally representing a direct bond or as a result of any one of the R¹⁴ to R¹⁸ substituents being optionally substituted with a second diazine formula. Illustrative of such diazine compounds are pyridazine, pyrimidine, pyrazine, and the like.

Illustrative heterocyclic nitrogen compounds that can be used as weakly basic amines in some embodiments of the present invention also include triazine compounds such as 1,3,5-triazines represented by the formula:

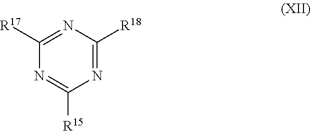

wherein in Formula (XII) above, R¹⁵, R¹⁷, and R¹⁸ are identical or different and each represents a hydrogen atom or a monovalent substituent. More specifically, said monovalent substituents R¹⁵, R¹⁷, and R¹⁸ in Formula (XII) above, may be the same as the monovalent substituents defined for Formulas (II) to (IV) above. It is to be further understood that Formulas (XII) is also intended to encompass compounds having two or more such triazine formulas, e.g., wherein two triazine formulas are directly bonded together as a result of any one of the R¹⁵, R¹⁷, and R¹⁸ substituents optionally representing a direct bond or as a result of any one of the R¹⁵, R¹⁷, and R¹⁸ substituents being optionally substituted with a second triazine formula. Illustrative of such triazines compounds are 1,3,5-triazine, and the like.

It is to be understood that the heterocyclic nitrogen compounds that can be used as weakly basic amines in some embodiments of the present invention contain at least one unfunctionalized nitrogen with a lone pair of electrons capable of forming a complex or adduct with the acid moiety. In other words, ionic ammonium salts such as described in U.S. Pat. No. 6,995,293 B2 (either alkylated or protonated) are not heterocyclic nitrogen stabilizing agents, since these quaternary ammonium salts do not have a free nitrogen lone pair.

Any of the R⁸ to R¹⁸ radicals of heterocyclic nitrogen compounds of Formulas (II) to (XII) above may be substituted if desired, with any suitable substituent containing from 1 to 30 carbon atoms that does not unduly adversely affect the desired result of the process or this invention. Substituents that may be on said radicals in addition of course to corresponding hydrocarbon radicals such as alkyl, aryl, aralkyl, alkaryl and cyclohexyl substituents, may include for example amino radicals such as —N(R¹⁹)₂; phosphine radicals such as -aryl-P(R¹⁹)₂; acyl radicals such as —C(O)R¹⁹ acyloxy radicals such as —OC(O)R¹⁹; amido radicals such as —CON(R¹⁹)₂ and —N(R¹⁹)COR¹⁹; sulfonyl radicals such as —SO₂R¹⁹, alkoxy radicals such as —OR¹⁹, sulfinyl radicals such as —SOR¹⁹, sulfenyl radicals such as —SR¹⁹, ionic radicals selected from the group consisting of: —SO₃M, —PO₃M, —N(R⁶)₃X¹ and —CO₂M as defined herein above for ionic phosphines, wherein M, X¹ and R⁶ are as defined above, as well as, nitro, cyano, trifluoromethyl, hydroxy radicals, and the like, wherein each R¹⁹ radical individually represents the same or different monovalent hydrocarbon radical having from 1 to 18 carbon atoms (e.g., alkyl, aryl, aralkyl, alkaryl and cyclohexyl radicals), with the proviso that in amino substituents such as —N(R¹⁹)₂ each R¹⁹ taken together can also represent a divalent bridging group that forms a heterocyclic radical with the nitrogen atom. Of course it is to be understood that any of the substituted or unsubstituted substituent radicals that make up a particular weakly basic amine may be the same or different.

Illustrative specific examples include imidazole and substituted imidazoles, such as 1-methylimidazole, 1-ethylimidazole, 1-n-propylimidazole, 1-isopropylimidazole, 1-butylimidazole, 2-methylimidazole, 2-ethylimidazole, 2-n-propylimidazole, 2-isopropylimidazole, 2-n-butylimidazole, 2-n-hexylimidazole, 2-n-heptylimidazole, 2-n-octylimidazole, 2-n-nonylimidazole, 2-n-decyl-imidazole, 2-n-undecylimidazole, 2-n-dodecylimidazole, 2-n-tridecylimidazole, 2-n-tetradecylimidazole, 2-n-pentadecylimidazole, 2-n-hexadecylimidazole, 2-n-heptadecylimidazole, 2-(2-ethylpentyl)imidazole, 2-ethyl-4-methylimidazole, 2-phenylimidazole, 1-benzyl-2-methylimidazole, 2,4,5-triphenylimidazole, 2-(2-propylhexyl)imidazole, 4-methylimidazole, 4-ethylimidazole, 3-n-propylimidazole, 4-isopropylimidazole, 4-butylimidazole, 4,5-dimethylimidazole, 4,5-diethylimidazole, 1-methyl-2-ethylimidazole, 1-methyl-4-ethylimidazole, 2-ethyl-4-methylimidazole, 1-benzyl-2-methylimidazole, 1-phenylimidazole, 2-phenylimidazole, 4-phenylimidazole, 2,4,5-triphenylimidazole, 1,2-trimethyleneimidazole, 1,5-trimethyleneimidazole, 4,5-trimethyleneimidazole, and the like, as well as, polar substituted imidazoles, such as e.g., 1-hydroxymethylimidazole, 2-hydroxymethylimidazole, 4-hydroxymethylimidazole, 1-(2-hydroxyethyl) imidazole, 2(2-hydroxyethyl)imidazole, 4-2(hydroxyethyl)imidazole, 1-carboxymethylimidazole, 2-carboxymethylimidazole, 4-carboxymethylimidazole, 1(2-carboxyethyl)imidazole, 4-(2-carboxyethyl) imidazole, 4-(2-carboxyethyl)imidazole, 4-(2-carboxy-2-hydroxyethyl) imidazole, and the like.

The preferred heterocyclic nitrogen compounds for use as weakly basic amines in some embodiments include benzimidazoles such as those represented by the formula:

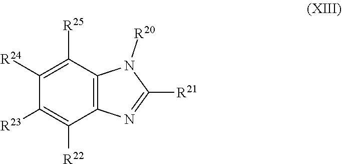

wherein in Formula (XIII) above R²⁰, R²¹, R²², R²³, R²⁴ and R²⁵ are identical or different and each represent a hydrogen atom or a monovalent substituent, provided R²⁰ and R²¹ are not both a monovalent hydrocarbon radical at the same time. More specifically said monovalent substituents of R²⁰, R²¹, R²², R²³, R²⁴ and R²⁵ may be the same as those monovalent substituents defined for Formulas (II) to (IV) above. Of course it is to be further understood that Formula (XIII) is also intended to encompass compounds having two or more such benzimidazole formulas, e.g., wherein two benzimidazole formulas are directly bonded together as a result of any one of the R²⁰ to R²⁵ substituents, e.g., R²¹, optionally representing a direct bond or as a result of any one of the R²⁰ to R²⁵ substituents, e.g., R²¹, being optionally substituted with a second benzimidazole formula, e.g., di-, bi-, or bis-benzimidazoles.

Examples of such benzimidazoles include benzimidazole and substituted benzimidazoles, such as 1-methylbenzimidazole, 1-ethylbenzimidazole, 1-n-propylbenzimidazole, 1-isopropylbenzimidazole, 1-butylbenzimidazole, 1-benzylbenzimidazole, 2-benzylbenzimidazole, 2-methylbenzimidazole, 2-ethylbenzimidazole, 2-n-propylbenzimidazole, 2-isopropylbenzimidazole, 2-n-butylbenzimidazole, 2-n-hexylbenzimidazole, 2-n-heptylbenzimidazole, 2-n-octylbenzimidazole, 2-n-nonylbenzimidazole, 2-n-decylbenzimidazole, 2-n-undecylbenzimidazole, 2-n-dodecylbenzimidazole, 2-n-tridecylbenzimidazole, 2-n-tetradecylbenzimidazole, 2-n-pentadecylbenzimidazole, 2-n-hexadecylbenzimidazole, 2-n-heptadecylbenzimidazole, 2-(2-ethylpentyl)benzimidazole, 2-(2-propylhexyl)benzimidazole, 2-phenylbenzimidazole, 2-phenylbenzimidazole, 1-benzylimidazole, 1-, cyclohexylbenzimidazole, 1-octylbenzimidazole, 1-dodecylbenzimidazole, 1-hexyldecylbenzimidazole, 5,6-dimethylbenzimidazole, 1-methyl-5,6-dimethylbenzimidazole, 4-methylbenzimidazole, 4-ethylbenzimidazole, 3-n-propylbenzimidazole, 4-isopropylbenzimidazole, 4-butylbenzimidazole, 4,5-dimethylbenzimidazole, 4,5-diethylbenzimidazole, 1-methyl-2-ethylbenzimidazole, 1-methyl-4-ethylbenzimidazole, 1-phenylbenzimidazole, and 4-phenylbenzimidazole, 5-bromobenzotriazole, 6-bromobenzotriazole, 5-chlorobenzotriazole, 6-chlorobenzotriazole, 5-chloro-1,6-dimethylbenzotriazole 5-chloro-6-methylbenzotriazole, 6-chloro-5-methylbenzotriazole, 5-chloro-6-methyl-1-phenylbenzotriazole, 4,5,6,7-tetrachlorobenzotriazole, 1-(2-iodoethyl)benzotriazole, 5-chloro-6-fluorobenzotriazole, 5-trifluoromethylbenzotriazole, 6-trifluoromethylbenzotriazole, and the like, as well as, polar substituted benzimidazoles, such as 1-acetylbenzimidazole, 1-benzoylbenzimidazole, 1-hydroxymethylbenzimidazole, 2-hydroxymethylbenzimidazole, 4-hydroxymethylbenzimidazole, 1-(2-hydroxyethyl)benzimidazole, 2(2-hydroxyethyl)benzimidazole, 4-2(hydroxyethyl)benzimidazole, 1-carboxymethylbenzimidazole, 2-carboxymethylbenzimidazole, 4-carboxymethylbenzimidazole, 1 (2-carboxyethyl)benzimidazole, 4-(2-carboxyethyl)benzimidazole, 4-(2-carboxyethyl)benzimidazole, 4-(2-carboxy-2-hydroxyethyl)benzimidazole, 1-ethyl-5,6-dimethylbenzimidazole, 1-isopropyl-5,6-benzimidazole, 1-isopropyl-5,6-benzimidazole, 5,6-dimethoxybenzimidazole, 4,5-trimethylenebenzimidazole, naphtho[1, 2-d]imidazole, naphtho[2,3-d]imidazole, 1-methyl-4-methoxybenzimidazole, 1-methyl-5-methoxybenzimidazole, 1-methyl-5,6-dimethoxybenzimidazole, and the like. Bi-, di- and bisbenzimidazoles are also included such as 2, 2′-ethylenebibenzimidazole, 2,2′-heptamethylenebibenzimidazole, 2,2′-hexamethylenebibenzimidazole, 2,2′(iminodiethylidene)-bibenzimidazole, 2,2′-(methyliminodiethylidene)bibenzimidazole, 2,2′-octamethylenebibenzimidazole, 2,2′pentamethylenebibenzimidazole, 2,2-p-phenylenebibenzimidazole, 2,2′-trimethylenebibenzimidazole, 2,2′-methylene bis(5,6-dimethylbenzimidazole), di-2-benzimidazolylmethane, 5,5′,6,6′-tetramethyl-2,2′-bibenzimidazole and 1,2-bis(5,6-dimethyl-2-benzimidazolyl)ethanol hydrochloride, and the like. The most preferred heterocyclic nitrogen compound of all is benzimidazole.

Because the non-volatile, weakly basic amine may condense on the VPH catalyst or have other undesirable downstream impacts including in refining and product alcohol purity, the amount of weakly basic amine that is volatilized and carried over with the volatilized aldehyde stream going to the VPH catalyst should be minimized Preferably, the amount of amine present in the vapor phase leaving the vaporization system should be less than 1 ppmw (by nitrogen). This level can be controlled by the amount of weakly basic amine added, the acid/amine ratio (i.e., avoiding high excess amine relative to moles of acid present), and the vaporization conditions (temperature, pressure, and purge rate). The amount of amine in the vapor phase leaving the vaporization system is determined by gas chromatography using techniques known to those having ordinary skill in the art based on the teachings herein.

As the aldehyde product is vaporized from the aldehyde feed to the vaporization system, the concentration of the non-volatilized components in the remaining material (e.g. the aldehyde heavies and weakly basic amine (and any acid adducts)), will increase accordingly. Thus the upper amount of the weakly basic amine that should be added is also governed by its solubility limit (and that of any acid adducts) in the non-volatilized liquid purge from the vaporization system. The solubility will depend on the vaporization separation temperature, as well as the particular amine itself. Alkanolamines may be preferable in some embodiments as they are usually liquid or low-melting solids of high solubility in aldehyde heavies streams at ambient temperatures.

The addition of the weakly basic amine to the aldehyde from which the aldehyde feed is to be vaporized may be carried out in any suitable manner desired. For instance, the weakly basic amine may be added to the aldehyde fluid that has been removed from a hydroformylation reaction zone and at any time prior to or during the vaporization of the aldehyde. However, since the weakly basic amine chosen to be used should not have any substantial detrimental affect on the aldehyde per se, the weakly basic amine may be added directly to the crude aldehyde immediately after a hydroformylation product-catalyst separation process. Indeed, it may be desirable to add the weakly basic amine, particularly heterocyclic nitrogen compounds, to the crude aldehyde as soon as possible so that the weakly basic amine is present right from the start of the hydrogenation process.

Regarding aldehyde feedstock that can be provided as liquid aldehyde stream in processes of the present invention, illustrative non-optically active aldehyde starting materials include propionaldehyde, n-butyraldehyde, isobutyraldehyde, n-valeraldehyde, 2-methyl 1-butyraldehyde, hexanal, hydroxyhexanal, 2-methyl valeraldehyde, heptanal, 2-methyl 1-hexanal, octanal, 2-methyl 1-heptanal, nonanal, 2-methyl-1-octanal, 2-ethyl 1-heptanal, 3-propyl 1-hexanal, decanal, adipaldehyde, 2-methylglutaraldehyde, 2-methyladipaldehyde, 3-methyladipaldehyde, 3-hydroxypropionaldehyde, 6-hydroxyhexanal, alkenals, e.g., 2-, 3- and 4-pentenal, and the like.

The alcohols resulting from embodiments of the present invention have a large number of uses including as solvents and raw materials for other products.

Some embodiments of the invention will now be described in more detail in the following Examples.

### EXAMPLES

All parts and percentages in the following examples are by weight unless otherwise indicated. Pressures are given as absolute pressure unless otherwise indicated.

The following examples are given to illustrate the invention and should not be construed as limiting its scope.

### Comparative Example A

Analysis of a butyraldehyde feed to a commercial-scale VPH vaporization system reveals a variety of butyric acid levels using conventional GC or titration methods. These levels range from 200 to 5000 ppmw (based on total organic feed). In addition, analysis of the resulting vaporized stream reveals that a significant amount of the feed acid was volatilized and still present in the feed going to the VPH catalyst. Vapor-Liquid Equilibirum (VLE) modelling indicates that the butyric acid should have gone down to the vaporizer heavies but for some reason, a significant portion was still passing on to the VPH bed.

### Comparative Example B

A sample of a zinc oxide (ZnO)-based VPH catalyst is exposed to butyric-acid containing vaporized aldehyde similar to the levels found in Comparative Example A under typical hydrogenation conditions. A solid is observed deposited on cooler downstream equipment presumably via sublimation from the catalyst. This solid is analyzed by TGA and FT-IR and found to be Zn(butyrate)₂ by comparison with authentic material.

### Comparative Example C

A sample of a ZnO-based VPH catalyst from a commercial-scale VPH reactor is examined by SEM and compared to catalyst that had not been charged to the reactor. Voids and new crystals are found within the body of the used catalyst; the latter were determined to be ZnO presumably from migration of volatile Zn(butyrate)₂ and subsequent hydrolysis to ZnO. The new crystals are clearly different (larger) than the original new catalyst indicative of a change in morphology of the catalyst support and the voids which would be consistent with a loss of catalyst integrity.

Comparative Examples A-C demonstrate the deleterious effect of butyric acid vapor on a heterogeneous catalyst.

### Inventive Example 1

A simple flash model in ASPEN for 1000 ppmw (94 ppmw by nitrogen) triethanolamine (a weakly basic amine) in a crude butyraldehyde stream with no butyric acid shows that approximately 1.4 ppmw (by nitrogen) of triethanolamine would be present in the vapor phase. This shows that only a small amount of triethanolamine leaves with the vaporized aldehyde stream. This would represent an upper limit because there would be less free amine in the vaporization system during operation as much of the weakly basic amine would be used in neutralizing acids to reduce the free acid to very low levels. The amount of triethanolamine in the vapor phase would thus be expected to be much less at the levels of free acid typically found in VPH feeds (typically 200-5000 ppmw) prior to neutralization. Based on this data, the amount of free triethanolamine in the overhead vapor stream can be calculated by reducing the feed to more closely match the moles of acidity present, thus neutralizing the free acid without impacting the VPH catalyst.

1. A process for the vapor phase hydrogenation of aldehydes comprising:
(a) providing a liquid aldehyde stream to a vaporization system to generate a vaporous aldehyde stream in the presence of a weakly basic amine, wherein the weakly basic amine has a normal boiling point that is at least 50° C. greater than the normal boiling point of the aldehyde, wherein the weakly basic amine reacts with acidic impurities in the liquid aldehyde stream to form ammonium salt adducts, and wherein the ammonium salt adducts and any excess weakly basic amine are removed as a heavies purge from the vaporization system;
(b) combining the vaporous aldehyde stream with a hydrogen stream by either providing a hydrogen stream to the vaporization system, by adding a hydrogen stream to the vaporous aldehyde stream following step (a), or by a combination thereof;
(c) providing the combined vaporous aldehyde and hydrogen stream to a vapor phase hydrogenation zone; and
(d) hydrogenating the vaporous aldehyde in the vapor phase hydrogenation zone.

2. The process of claim 1, wherein the weakly basic amine comprises a trialkanolamine or an imidazole.

3. The process of claim 2, wherein the weakly basic amine comprises triethanolamine or benzimidazole.

4. The process of claim 1, wherein the weakly basic amine concentration in the combined vaporous aldehyde and hydrogen stream in step (c) is less than 1 ppmw (by nitrogen).

5. The process of claim 1, further comprising measuring the acid content of the liquid aldehyde stream, wherein the amount of weakly basic amine added to the vaporization system is between 0.1 and 5 equivalents of weakly basic amine to equivalents of acid.

6. The process of claim 1, wherein the liquid aldehyde stream is provided from a hydroformylation reaction and a product-catalyst separation step, wherein hydroformylation catalyst is separated from a hydroformylation product stream in the product-catalyst separation step to provide the liquid aldehyde stream.

7. The process of claim 1, wherein the basicity of the weakly basic amine, determined as the pKa of the conjugate acid, is from 5 to 11 at a temperature of 25° C.

8. The process of claim 1, wherein the weakly basic amine has a normal boiling point that is at least 100° C. greater than the normal boiling point of the aldehyde.
